(19) United States
(12) Patent Application Publication
(10) Pub. No.: US 2026/0191642 A1
(43) Pub. Date: Jul. 9, 2026

(54) AORTIC DOCKING STATION

(71) Applicant: Edwards Lifesciences Corporation, Irvine, CA (US)

(72) Inventors: Michael G. Valdez, Riverside, CA (US); Tiana (formerly Thu Thi Anh) Tran, Westminster, CA (US); Omar Fawzi Azanki, Coto de Caza, CA (US)

(21) Appl. No.: 19/559,803

(22) Filed: Mar. 6, 2026

Related U.S. Application Data
(63) Continuation of application No. PCT/US2024/045594, filed on Sep. 6, 2024.
(60) Provisional application No. 63/581,963, filed on Sep. 11, 2023.

(51) Int. Cl. A61F 2/24

(52) U.S. Cl. CPC A61F 2/2418; A61F 2/2433; A61F 2220/0008; A61F 2230/0013; A61F 2230/0054; A61F 2230/0093; A61F 2250/0063; A61F 2250/0069

(57) ABSTRACT

A docking station for use in aortic valve replacement comprises an inner wire frame forming a valve seat configured to receive or couple to an artificial valve. The docking station may include an outer wire frame that may be joined to the inner wire frame at one or more apices, and the outer wire frame may include a series of cells about a circumference of the outer wire frame. The docking station may include a sealing skirt that extends from the valve seat to the apices and turns upward along an exterior region of the outer wire frame. The docking station may include a ratio of a width to a length of the docking station includes a ratio greater than 1:1 and less than 3:1.

### CROSS REFERENCE TO RELATED APPLICATION

This application is a continuation of PCT Patent Application No. PCT/US2024/045594 filed on September 6, 2024, which application claims priority to and the benefit of U.S. Provisional Patent Application Serial No. 63/581,963, filed on September 11, 2023, each of these applications being incorporated by this reference herein in its entirety.

### FIELD

The present disclosure relates to an aortic docking station designed to accommodate and/or include an artificial valve to replace a native aortic valve.

### BACKGROUND

Unless otherwise indicated, the materials described herein are not prior art to the claims in the present application and are not admitted to be prior art by inclusion in this section.

The human heart may suffer from various valvular diseases. These valvular diseases may result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally invasive surgical approaches are used in various procedures to deliver prosthetic medical devices to locations inside the body that are not readily accessible by surgery or where access without surgery is desirable. Some of the devices used in these procedures include guidewires, catheters, guide sheaths, and the like, which aid in the insertion of a prosthetic heart valve inside the body.

Prosthetic heart valves may also be used to treat cardiac valvular disorders. The native heart valves (the aortic, pulmonary, tricuspid, and mitral valves) function to prevent backward flow or regurgitation, while allowing forward flow. These heart valves may be rendered less effective by congenital, inflammatory, infectious conditions, etc. Such conditions may eventually lead to serious cardiovascular compromise or death. For example, in aortic insufficiency, the aortic valves do not function properly and may cause regurgitation. For many years, doctors attempted to treat such disorders with surgical repair or replacement of the valve during open heart surgery.

A transcatheter technique for introducing and implanting a prosthetic heart valve using a catheter in a manner that is less invasive than open heart surgery may reduce complications associated with open heart surgery. In this technique, a prosthetic valve may be mounted in a crimped state on the end portion of a catheter and advanced through a blood vessel of the patient until the valve reaches the implantation site. The valve at the catheter tip may then be expanded to its functional size at the site of the defective native valve, such as by inflating a balloon on which the valve is mounted or, for example, the valve may have a resilient, self-expanding stent or frame that expands the valve to its functional size when it is advanced from a delivery sheath at the distal end of the catheter. Optionally, the valve may have a balloon-expandable frame, self-expanding frame, a mechanically-expandable frame, and/or a frame expandable in multiple or a combination of ways.

Transcatheter heart valves (THVs) may be appropriately sized for placement inside many native cardiac valves or orifices. However, with larger native valves, blood vessels (e.g., an enlarged aorta), grafts, etc., aortic transcatheter valves might be too small to secure into the larger implantation or deployment site. In this case, the transcatheter valve may not be large enough to sufficiently expand inside the native valve or other implantation or deployment site or the implantation/deployment site may not provide a good seat for the THV to be secured in place. As one example, aortic insufficiency may be associated with difficulty securely implanting a THV in the aorta and/or aortic valve. Replacement of the aortic valves may pose challenges in that replacement valves may not fit properly due to the various aortic sizes.

Additionally, blockage of the coronary ostia is a concern. Acute coronary obstruction, i.e., ostia or ostium blockage, is a potential complication of transcatheter aortic valve replacement. Left coronary ostial obstruction is much more common compared to right coronary occlusion due to its relatively lower ostial height from the aortic annulus. Accordingly, there exists a need for an aortic docking station designed to accommodate standardized sizes of heart valves and that reduces or eliminates coronary ostium blockage.

The subject matter claimed herein is not limited to implementations that solve any disadvantages or that operate only in environments such as those described above. Rather, this background is only provided to illustrate one example area where some implementations described herein may be practiced.

### SUMMARY

This summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter. Certain embodiments of the disclosure pertain to docking stations, frame adaptors, prestents, and the like for engaging and retaining a prosthetic implant such as a prosthetic heart valve in a lumen of the body, such as a blood vessel or valve of the heart.

In a representative embodiment of a docking station for use in aortic valve replacement, the docking station may include an inner wire frame forming a valve seat configured to receive or couple to an artificial valve. The docking station may include an outer wire frame that may be joined to the inner wire frame at one or more apices, and the outer wire frame may include a series of cells about a circumference of the outer wire frame. The docking station may include a sealing skirt that extends from the valve seat to the apices and turns upward along an exterior region of the outer wire frame. The docking station may include a ratio of a width to a length of the docking station that includes a ratio greater than 1:1 and less than 3:1.

In a representative method for reducing ostium blockage, the method may include implanting a docking station, the docking station may include an inner wire frame forming a valve seat configured to receive or couple to an artificial valve. The docking station may include an outer wire frame that may be joined to the inner wire frame at one or more apices, and the outer wire frame may include a series of cells about a circumference of the outer wire frame. The docking station may include a sealing skirt that extends from the valve seat to the apices and turns upward along an exterior region of the outer wire frame. The docking station may include a ratio of a width to a length of the docking station that includes a ratio greater than 1:1 and less than 3:1.

In a representative method, the method may include guiding a delivery catheter through a patient to a native aortic valve of the patient; deploying a docking station within the native aortic valve, the docking station compatible with or consistent with any of the implants or docking stations of the present disclosure; guiding a procedure catheter through the patient to the docking station through the outer wire frame, and into an ostia downstream from the native aortic valve; and performing a procedure via the procedure catheter in the ostia.

The foregoing and other objects, features, and advantages of the described technology will become more apparent from the following detailed description, which proceeds with reference to the accompanying figures. The features and advantages of the invention may be realized and obtained by means of the instruments and combinations particularly pointed out in the appended claims, or may be learned by the practice of the invention as set forth herein.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 schematically illustrates a first stage in an example aortic valve replacement procedure where a delivery catheter and a guidewire are inserted into a blood vessel of a patient and navigated through the blood vessel and into the aortic arch of the heart of the patient, towards a native aortic valve of the heart.

FIG. 2A schematically illustrates a second stage in the example aortic valve replacement procedure where the delivery catheter extends through the native aortic valve and arrives at a target or deployment location.

FIG. 2B schematically illustrates a third stage in the example aortic valve replacement procedure where a docking station is fully implanted at the native aortic valve of the patient.

FIG. 2C schematically illustrates a fourth stage in the example aortic valve replacement procedure where a prosthetic heart valve delivery apparatus has deployed a prosthetic heart valve in the implanted docking station at the native aortic valve.

FIG. 2D schematically illustrates a sixth stage in the example aortic valve replacement procedure where the delivery catheter and the guidewire have been removed from the patient.

FIG. 3A is a cutaway view of the human heart in a diastolic phase.

FIG. 3B is a cutaway view of the human heart in a systolic phase.

FIG. 4A is a cutaway view of the human heart with an example docking station positioned in the aortic valve.

FIG. 4B is an end view of an example docking station and valve showing the valve in an open configuration such that blood may flow through the valve, e.g., when the heart is in a diastolic phase.

FIG. 4C is an end view of the docking station and valve of FIG. 4B showing the valve in a closed configuration, e.g., when the heart is in a systolic phase.

FIGS. 5A and 5B schematically illustrate deployment of a docking station.

FIGS. 5C and 5D schematically illustrate deployment of a valve in the docking station.

FIG. 6 illustrates a front view of an example docking station configured to dock and/or support one or more prosthetic valves and/or valve components.

FIG. 7 illustrates a front view of another example docking station configured to dock and/or support one or more prosthetic valves and/or valve components.

FIG. 8 illustrates a top view of the docking station of FIG. 7.

FIG. 9 illustrates a cutaway side view of the docking station of FIG. 7 with a valve disposed therein and implanted in a patient to replace a native aortic valve.

FIGS. 10 illustrates an additional example docking station with radio opaque markers and horizontal reinforcing struts.

FIG. 11 illustrates a procedure catheter for performing a procedure through a docking station deployed within a patient.

FIG. 12 illustrates a partial section view of the docking station of FIG. 7.

FIG. 13A illustrates a partial section view of a docking station including a retaining barb.

FIG. 13B illustrates a front view of a portion of the docking station of FIG. 13A.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

### Explanation of Terms

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

It should be understood that the disclosed embodiments may be adapted for delivery and implantation in any of the native annuluses and blood vessels of the heart (e.g., the pulmonary, mitral, and tricuspid annuluses, the inferior and superior vena cava, etc.), and may be used with any of various delivery approaches (e.g., retrograde, antegrade, transseptal, transventricular, transatrial, etc.).

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods may be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, may be used in combination with any other feature described herein.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language. As used herein, the term “and/or” used between the last two of a list of elements means any one or more of the listed elements. For example, the phrase “A, B, and/or C” means “A”, “B,”, “C”, “A and B”, “A and C”, “B and C”, or “A, B, and C.”

In the context of the present application, the terms “lower” and “upper” are used interchangeably with the terms “inflow” and “outflow,” respectively. Thus, for example, typically the lower end of a valve or docking station as depicted in the figures is its inflow end and the upper end of the valve or docking station is its outflow end unless explicitly described otherwise.

As used herein, the term “proximal” refers to a position, direction, or portion of a device that is closer to the user and further away from the implantation site and/or body lumen orifice. As used herein, the term “distal” refers to a position, direction, or portion of a device that is further away from the user and closer to the implantation site and/or body lumen orifice. Thus, for example, proximal motion of a device is motion of the device toward the user, while distal motion of the device is motion of the device away from the user.

The terms “longitudinal” and “axial” refer to an axis extending in the upstream and downstream directions, or in the proximal and distal directions, unless otherwise expressly defined.

Although there are alternatives for various components, features, parameters, operating conditions, etc., set forth herein, that does not mean that those alternatives are necessarily equivalent and/or perform equally well. Nor does it mean that the alternatives are listed in a preferred order unless stated otherwise.

Directions and other relative references (e.g., inner, outer, upper, lower, etc.) may be used to facilitate discussion of the drawings and principles herein, but are not intended to be limiting. For example, certain terms may be used such as “inside,” “outside,”, “top,” “down,” “interior,” “exterior,” and the like. Such terms are used, where applicable, to provide some clarity of description when dealing with relative relationships, particularly with respect to the illustrated embodiments. Such terms are not, however, intended to imply absolute relationships, positions, and/or orientations. For example, with respect to an object, an “upper” part may become a “lower” part simply by turning the object over. Nevertheless, it is still the same part and the object remains the same. As used herein, “and/or” means “and” or "or", as well as “and” and “or”.

As used herein, the terms “integrally formed” and “unitary construction” refer to a construction that does not require any sutures, fasteners, or other securing means to attach two portions of the construction together.

### EXAMPLES OF DISCLOSED TECHNOLOGY

The present disclosure pertains to valve adapter/docking station/landing zone/prestent technology for implanting a prosthetic heart valve, such as a transcatheter heart valve, in a lumen or valve of the heart where the diameter of the lumen or valve is significantly greater than the functional diameter of the prosthetic valve. In certain examples, the docking station may comprise a radially expandable and collapsible frame formed from a plurality of struts, and including a valve seat within the frame configured to receive an expandable prosthetic valve. In certain embodiments, the valve seat may comprise a plurality of struts coupled to the frame and angled inwardly toward the longitudinal axis of the frame. The valve seat may be configured to engage and retain prosthetic valves of a variety of types and sizes. The outer aspect of the docking station frame may engage the surrounding tissue of the native lumen and form a seal, and the valve seat may engage and retain the prosthetic heart valve within the docking station. In certain embodiments, the frame may comprise a sealing member (e.g., a sealing skirt) configured to form a seal between the frame and the surrounding anatomy without substantially interfering with blood flow entering the downstream portions of the frame, such as adjacent the ostia of the hepatic veins when implanted in the aortic valve.

In certain embodiments, the struts of the valve seat may form valve seat frame cells of the frame. In certain embodiments, struts and/or cells of the valve seat may comprise free end portions/apices, which may be disposed within the lumen of the docking station frame and define a reduced diameter portion configured to engage and retain a prosthetic heart valve. In certain embodiments, the struts of the valve seat may be coupled to the docking station frame at frame junctions, and the free end portions/apices of the valve seat may be offset from the frame junctions in an upstream direction toward the outflow end of the frame. This reduces or minimizes the length of the prosthetic valve that protrudes or extends distally or in the upstream direction from the docking station. In certain embodiments, the struts of the valve seat may be wholly disposed within the docking station frame, or the free end portions/apices of the valve seat may define an upstream-most end of the docking station frame.

In certain embodiments, the docking station frame may comprise a plurality of circumferentially-arranged longitudinal struts. The longitudinal struts may reduce or prevent foreshortening of the frame between the collapsed and expanded configuration. This may facilitate more accurate and/or predictable deployment of the docking station from the collapsed delivery configuration. The longitudinal struts may also facilitate recapture of the docking station frame from a partially deployed state by limiting an angle formed by the flared, partially deployed portion of the frame and the longitudinal axis of the delivery apparatus. The longitudinal struts may also strengthen the frame and reduce or eliminate infolding or invagination of the frame during recapture.

In certain embodiments, the docking station frame may comprise a plurality of free end portions or apices arranged circumferentially around the frame. In certain embodiments, the free apices may be located between pairs of adjacent longitudinal struts. In certain embodiments, the free apices may be proximal and/or distal apices of frame cells defined between pairs of longitudinal frame struts. In certain embodiments, the frame cells may be axially spaced apart from each other. The free apices may be configured to engage the surrounding tissue of a body lumen in which the docking station frame is implanted to prevent frame movement/migration/rotation relative to the body lumen.

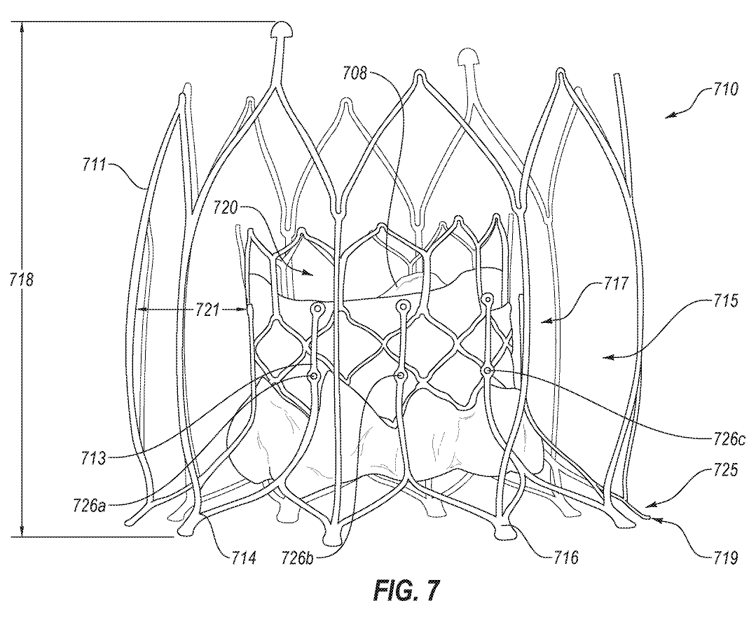

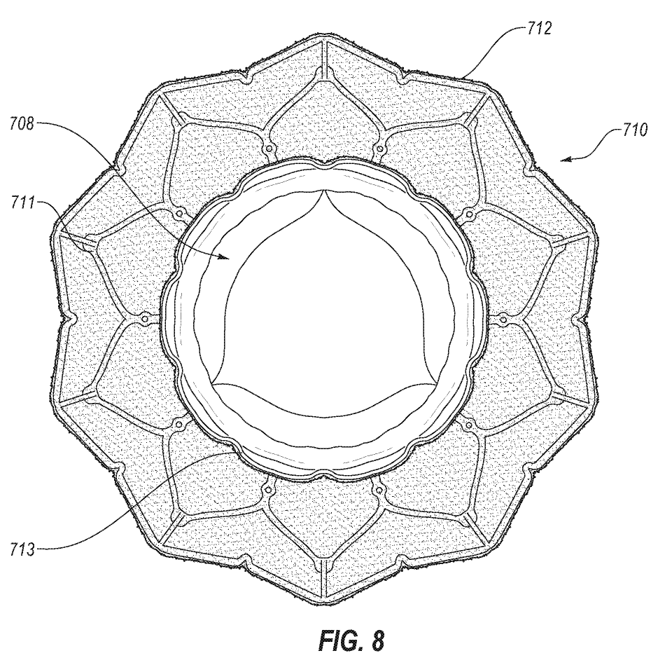

In some example embodiments, docking stations/devices for prosthetic valves or THVs are illustrated as being used within the superior vena cava (SVC), inferior vena cava (IVC), or both the SVC and the IVC, although the docking stations/devices (e.g., the aortic docking station as described herein, e.g., in FIGS. 7-9, other docking stations/devices described herein, modified versions of the docking stations, etc.) may be used in other areas of the anatomy, heart, or vasculature, such as the aortic valve, the aorta, tricuspid valve, the pulmonary valve, the pulmonary artery, the mitral valve, or other locations. The docking stations/devices described herein may be configured to compensate for the deployed transcatheter valve or THV being smaller and/or having a different geometrical shape than the space (e.g., anatomy/heart/vasculature/etc.) in which it is to be placed. For example, the native anatomy (e.g., the IVC) may be oval, egg shaped, or another shape, while the prosthetic valve or THV may be cylindrical.

Various embodiments of docking stations/devices and examples of prosthetic valves or transcatheter valves are disclosed herein, and any combination of these options may be made unless specifically excluded. For example, any of the docking stations/devices disclosed, may be used with any type of valve, and/or any delivery system, even if a specific combination is not explicitly described. Likewise, the different constructions and features of docking stations/devices and valves may be mixed and matched, such as by combining any docking station type/feature, valve type/feature, covering/sealing element, etc., even if not explicitly disclosed. In short, individual components of the disclosed systems may be combined unless mutually exclusive or physically impossible.

For the sake of uniformity, in the present disclosure the docking stations are typically depicted such that the right atrium end (e.g., the outflow end) is up, while the ventricular end or IVC end (e.g., the inflow end) is down unless otherwise indicated.

### First Representative Embodiment

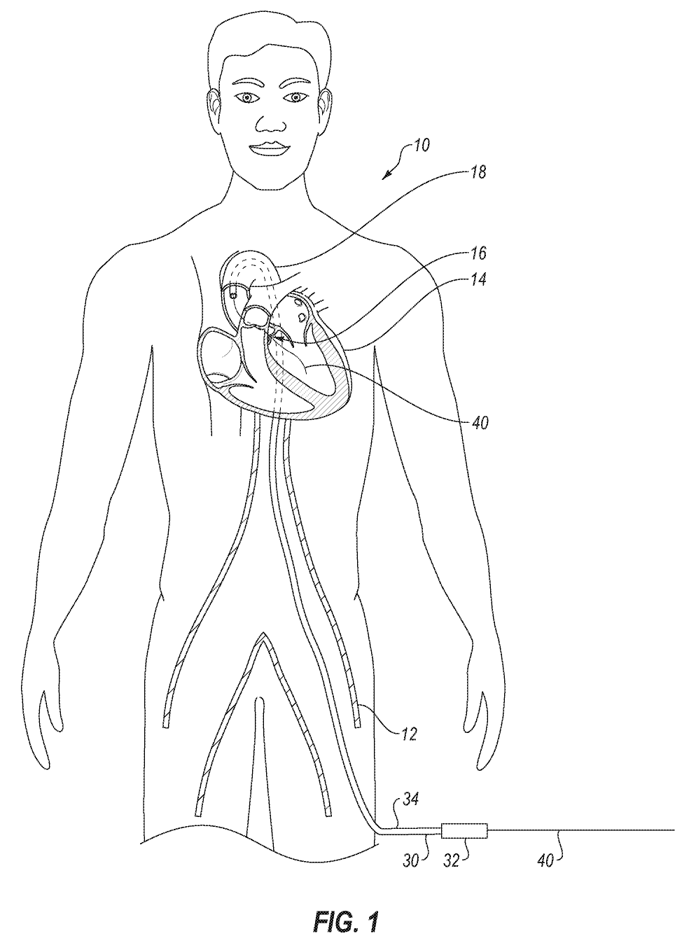

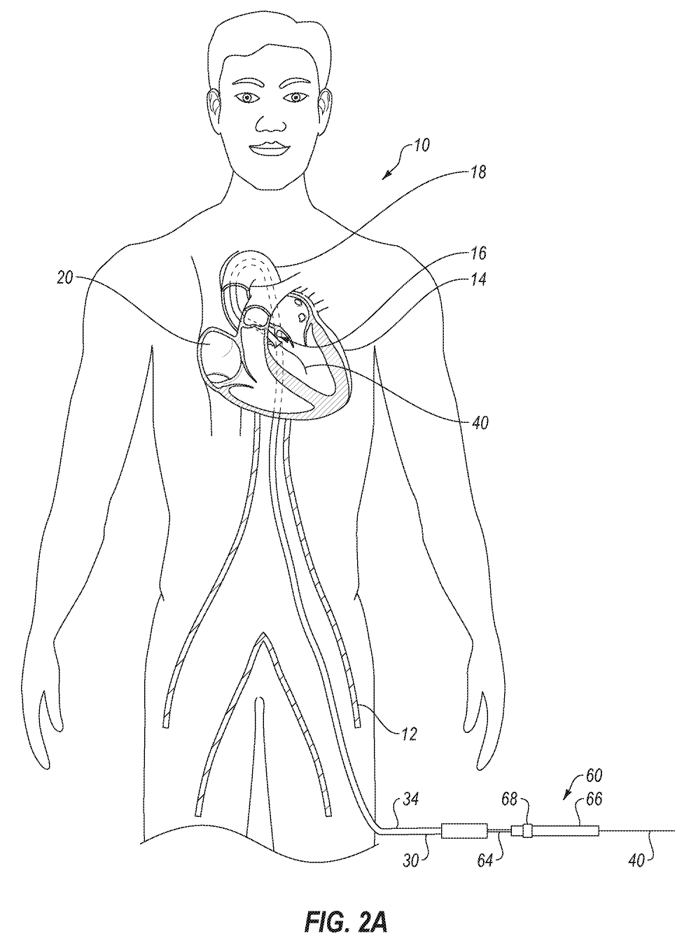

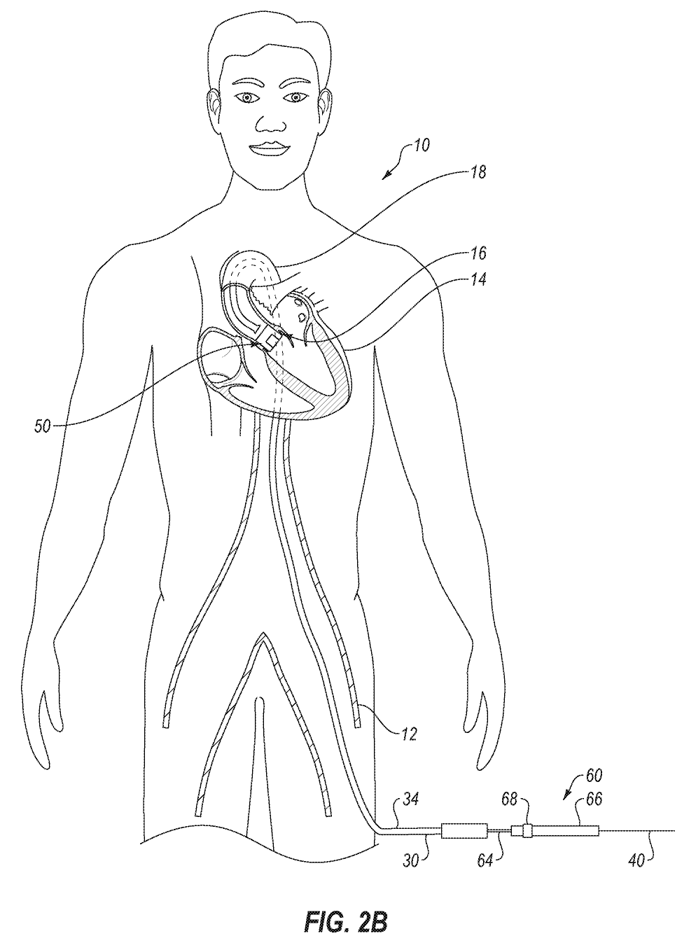

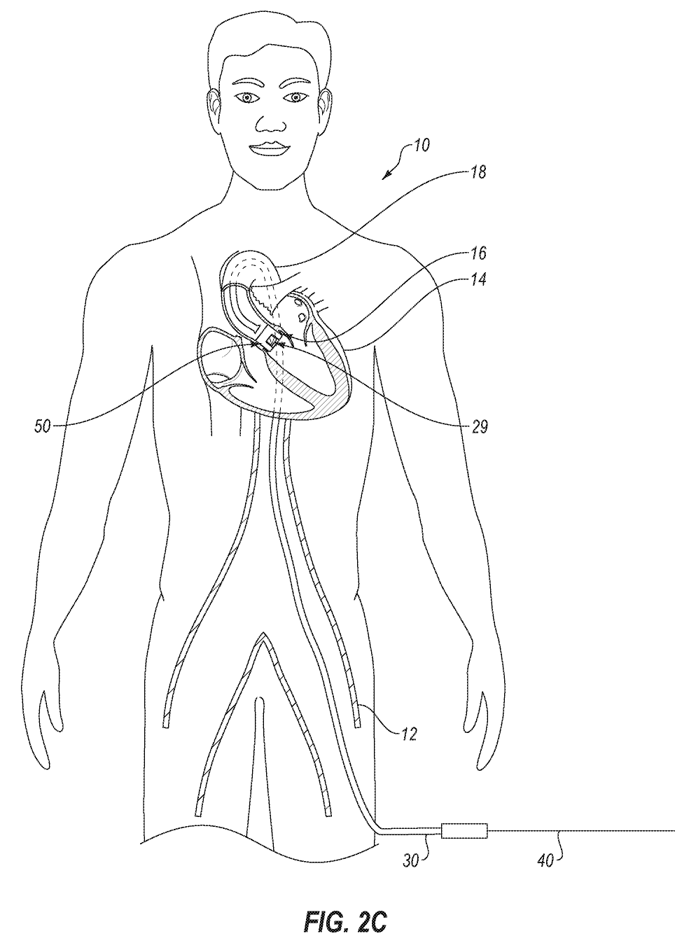

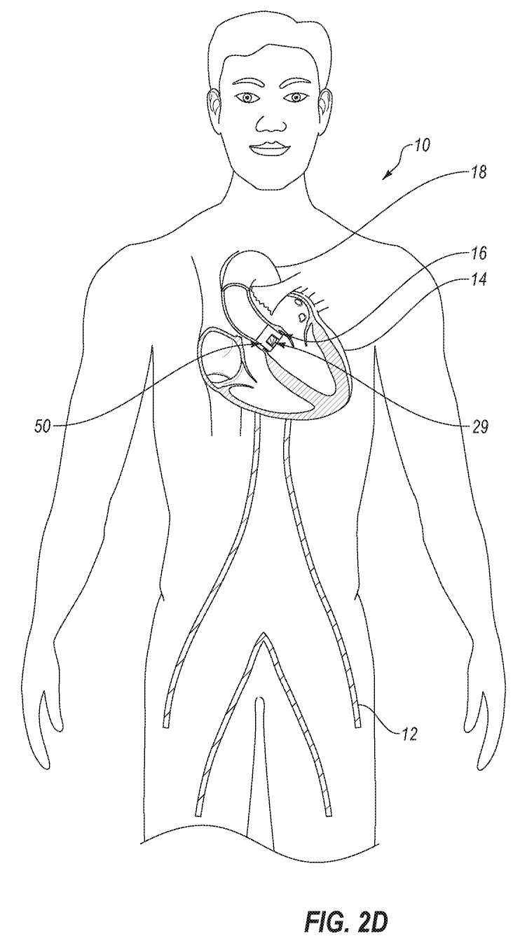

FIGS. 1-2D depict an example transcatheter heart valve replacement procedure (e.g., an aortic valve replacement procedure) which utilizes a docking device 52 and a prosthetic heart valve 62, according to one example. During the procedure, a clinician first creates a pathway to a patient’s native heart valve using a guidewire 40 (FIG. 1). The clinician then positions the delivery catheter 30 to the target position (e.g., the aortic valve 16) (FIG. 2A) and delivers and implants the docking station 50 at the patient’s native aortic valve 16 (FIG. 2B). The clinician then implants the prosthetic heart valve 29 within the implanted docking station 50 using a prosthetic valve delivery apparatus 60 (FIGS. 2B-2C). Thereafter, the clinician removes the prosthetic valve delivery apparatus 60 and the delivery catheter 30 from the patient 10 (FIG. 2D.

FIG. 1 depicts a first stage in an aortic valve replacement procedure, according to one example, where the delivery catheter 30 and a guidewire 40 are inserted into a blood vessel 12 of a patient 10 and navigated through the blood vessel 12, into an aortic arch 18 of the patient 10, and toward the native aortic valve 16. Together, the delivery catheter 30 and the guidewire 40 may provide a path for the docking station 50, and for the prosthetic valve delivery apparatus 60, to be navigated past the aortic arch 18 and to the target or implantation site, the native aortic valve 16 or native aortic valve annulus.

Initially, the clinician may make an incision in the patient’s body to access the blood vessel 12. For example, the clinician may make an incision in the patient’s groin to access a femoral artery. Thus, in some examples, the blood vessel 12 may be a femoral artery.

After making the incision at the blood vessel 12, the clinician may insert the delivery catheter 30, the guidewire 40, and/or additional devices (such as an introducer device) through the incision and into the blood vessel 12. The delivery catheter 30 is configured to facilitate the carrying and/or delivery of the docking station 50 into and through the blood vessel 12 and may extend through the blood vessel 12, into the aortic arch 18, and to the native aortic valve 16. The delivery catheter 30 may comprise a handle 32 and a shaft 34 extending distally from the handle 32. The shaft 34 may extend through the blood vessel 12 and into the heart 14 while the handle 32 remains outside the body of the patient 10 and may be operated by the user in order to manipulate the shaft 34 (FIG. 1).

The guidewire 40 is configured to guide the delivery apparatuses (e.g., the delivery catheter 30, the prosthetic valve delivery apparatus 60, additional catheters, or the like) and their associated devices (e.g., docking station 50, prosthetic heart valve 29, and the like) to the implantation site within the heart 14, and thus may extend all the way through the blood vessel 12, into the aortic arch 18, and into the native aortic valve 16 of the heart 14 (FIG. 1).

In some instances, an introducer device may be inserted through a lumen of the delivery catheter 30 prior to inserting the delivery catheter 30 into the blood vessel 12. In some instances, the introducer device may include a tapered end that extends out a distal tip of the delivery catheter 30 and that is configured to guide the delivery catheter 30 into the aortic valve 16 annulus over the guidewire 40. Additionally, in some instances the introducer device may include a proximal end portion that extends out a proximal end of the delivery catheter 30. Once the delivery catheter 30 reaches the aortic valve 16 annulus and the docking station 50 has been implanted in the aortic valve 16, the clinician may remove the introducer device from inside the delivery catheter 30 and the patient 10. Thus, only the delivery catheter 30 and the guidewire 40 may remain inside the patient 10.

FIG. 2A depicts a second stage in the example aortic valve replacement procedure where the delivery catheter has reached the target or implantation site. For example, the delivery catheter 30 may reach the aortic valve 16 and/or an annulus associated therewith.

After the delivery catheter 30 is positioned within the aortic valve 16, the clinician may deploy the docking station 50 into the aortic valve. For example, an outer sheath of the delivery catheter may be retracted such that the docking station 50 is exposed and may expand from a compressed state into an expanded state.

In some examples, the docking station 50 may be constructed from, formed of, and/or comprise a shape memory material, and as such, may return to its original, pre-formed shape when the sheath of the delivery catheter 30 is retracted and is no longer constrained by the sheath. Additionally or alternatively, a balloon (not illustrated) may be included radially inward from the docking station 50 within the delivery catheter 30 and after exposing the docking station 50, the balloon may be inflated (by liquid or air) to cause the docking station 50 to expand from its compressed state to the expanded state.

After deploying and implanting the docking station 50 at the native aortic valve 16, the clinician may disconnect the docking station 50 from the delivery catheter 30. Once the docking station 50 is disconnected from the delivery catheter 30, the clinician may retract the delivery catheter 30 out of the blood vessel 12 and away from the patient 10 so that the clinician may deliver and implant a prosthetic heart valve 29 within the implanted docking station 50 at the native aortic valve 16.

FIG. 2B depicts the third stage in the aortic valve replacement procedure, where the docking station 50 has been fully deployed and implanted at the native aortic valve 16. In some examples, the delivery catheter may facilitate delivery of the artificial valve 29. Additionally or alternatively, a separate delivery device (such as the valve delivery apparatus 60) may be used to facilitate delivery of the valve 29.

As shown in FIG. 2B, the prosthetic valve delivery apparatus 60 may include a delivery shaft 64 and a handle 66, the delivery shaft 64 extending distally from the handle 66. The delivery shaft 64 is configured to extend into the patient’s vasculature to deliver, implant, expand, and/or otherwise deploy the prosthetic heart valve 29 within the docking station 50 at the native aortic valve 16. The handle 66 is configured to be gripped and/or otherwise held by the user to advance the delivery shaft 64 through the patient’s vasculature.

In some examples, the handle 66 may comprise one or more articulation members 68 that are configured to aid in navigating the delivery shaft 64 through the blood vessel 12 and the heart 14. Specifically, the articulation member(s) 68 may comprise one or more of knobs, buttons, wheels, and/or other types of physically adjustable control members that are configured to be adjusted by the user to flex, bend, twist, turn, and/or otherwise articulate a distal end portion of the delivery shaft 64 to aid in navigating the delivery shaft 64 through the blood vessel 12 and to the docking station 50 at the aortic valve 16.

In some examples, the prosthetic valve delivery apparatus 60 may include an expansion mechanism that is configured to radially expand and deploy the prosthetic heart valve 29 at the implantation site. In some instances, the expansion mechanism may include an inflatable balloon that is configured to be inflated to radially expand the prosthetic heart valve 29 within the docking device 52. The inflatable balloon may be coupled to the distal end portion of the delivery shaft 64.

In other examples, the prosthetic heart valve 29 may be self-expanding and may be configured to radially expand on its own upon removal of a sheath or capsule covering the radially compressed prosthetic heart valve 29 on the distal end portion of the delivery shaft 64. In still other examples, the prosthetic heart valve 29 may be mechanically expandable and the prosthetic valve delivery apparatus 60 may include one or more mechanical actuators (e.g., the expansion mechanism) configured to radially expand the prosthetic heart valve 29.

FIG. 2C depicts the fourth stage in the aortic valve replacement procedure where the prosthetic heart valve 29 (which may also be referred to herein as a “transcatheter heart valve” or “THV” for short, “replacement heart valve,” and/or “prosthetic aortic valve”) has been deployed within the docking station 50.

FIG. 2D shows a fifth stage in the aortic valve replacement procedure where the prosthetic heart valve 29 is in its radially expanded configuration and implanted within the docking station 50 in the native aortic valve 16. As shown in FIG. 2D, the prosthetic heart valve 29 is received and retained within the docking station 50. Thus, the docking station 50 aids in anchoring the prosthetic heart valve 29 within the native aortic valve 16. Additionally, the guidewire 40 and the delivery catheter 30 have been removed from the patient 10.

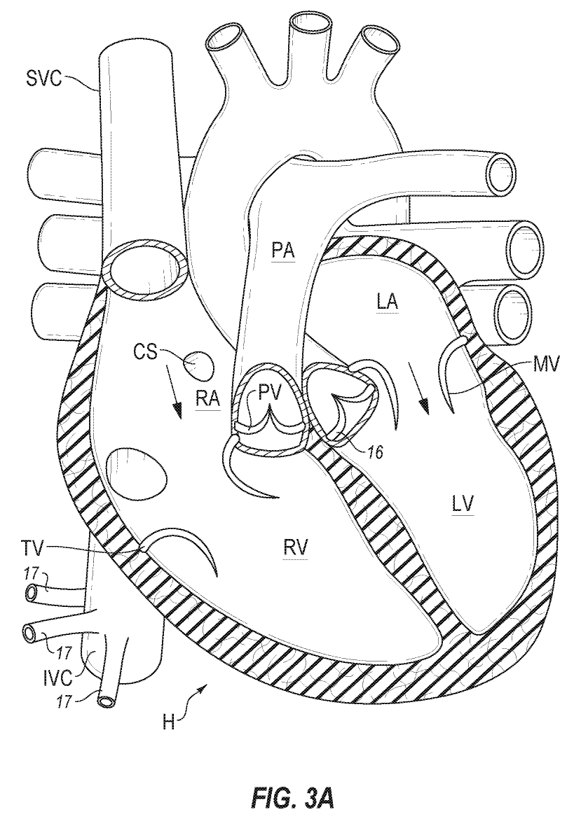

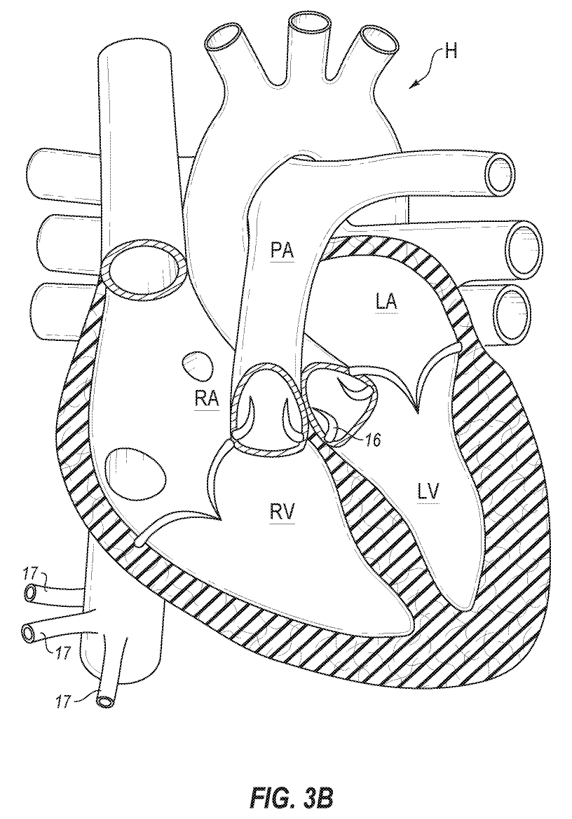

FIGS. 3A and 3B are cutaway views of the human heart (H) in diastolic and systolic phases, respectively. The right ventricle (RV) and left ventricle (LV) are separated from the right atrium (RA) and left atrium (LA), respectively, by the tricuspid valve (TV) and the mitral valve (MV); i.e., the atrioventricular valves. Additionally, the aortic valve (AV) separates the left ventricle (LV) from the ascending aorta (not identified) and the pulmonary valve (PV) separates the right ventricle from the pulmonary artery (PA). Each of these valves has flexible leaflets extending inward across the respective orifices that come together or “coapt” in the flowstream to form one-way, fluid-occluding surfaces. The docking stations and valves of the present application are described, for illustration, primarily with respect to the inferior vena cava (IVC), superior vena cava (SVC), and aorta/aortic valve 16. A defective aortic valve 16, for example, may be a stenotic aortic valve and/or suffer from insufficiency and/or regurgitation. The blood vessels, such as the aorta, IVC, SVC, pulmonary artery, may be healthy or may be dilated, distorted, enlarged, have an aneurysm, or be otherwise impaired. Anatomical structures of the right atrium RA, right ventricle RV, left atrium LA, and left ventricle LV will be explained in greater detail. The devices described herein may be used in various areas whether explicitly described herein or not, e.g., in the IVC and/or SVC, in the aorta (e.g., an enlarged aorta) as treatment for a defective aortic valve, in other areas of the heart or vasculature, in grafts, etc.

The right atrium RA receives deoxygenated blood from the venous system through the superior vena cava SVC and the inferior vena cava IVC, the former entering the right atrium from above, and the latter from below. The hepatic veins 17 carry blood from the liver to the inferior vena cava (IVC). The coronary sinus CS is a collection of veins joined together to form a large vessel that collects deoxygenated blood from the heart muscle (myocardium), and delivers it to the right atrium RA. During the diastolic phase, or diastole, seen in FIG. 3A, the deoxygenated blood from the IVC, SVC, and CS that has collected in the right atrium RA passes through the tricuspid valve TV and into the RV as the right ventricle RV expands. In the systolic phase, or systole, seen in FIG. 3B, the right ventricle RV contracts to force the deoxygenated blood collected in the RV through the pulmonary valve PV and pulmonary artery into the lungs.

The left atrium LA receives oxygenated blood from the left and right pulmonary veins, which then travels through the mitral valve to the left ventricle. During the diastolic phase, or diastole, seen in FIG. 3A, the oxygen rich blood that collects in the left atrium LA passes through the mitral valve MV and into the left ventricle LV as the left ventricle LV expands. In the systolic phase, or systole, seen in FIG. 3B, the left ventricle LV contracts to force the oxygen rich blood through the aortic valve AV and aorta into the body through the circulatory system. In certain embodiments, the devices described herein may be used to supplement or replace the function of a defective aortic valve. For example, the devices herein may be particularly effective for treating aortic insufficiency. During diastole, the leaflets of a normally functioning aortic valve AV close to prevent the oxygen rich blood from regurgitating back into the left ventricle LV. When the aortic valve does not operate normally, blood backflows or regurgitates into the left ventricle LV. A THV implanted in the aortic valve helps prevent or inhibit blood from back-flowing into the left ventricle LV during the diastole phase. The length L, diameter, D, and curvature or contour of the aortic root may vary greatly between different patients, especially if the aorta is a dilated, distorted, or enlarged. Further, the size or diameter D may vary significantly along the length L of an individual aorta.

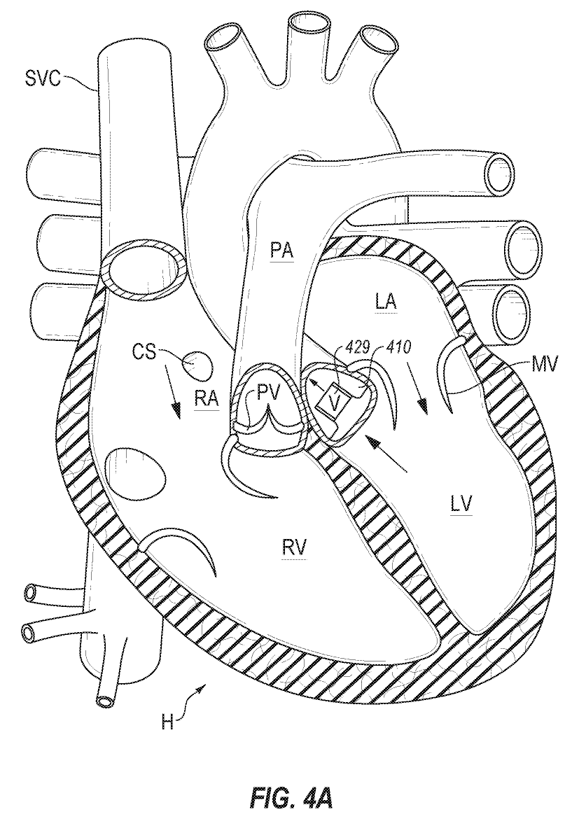

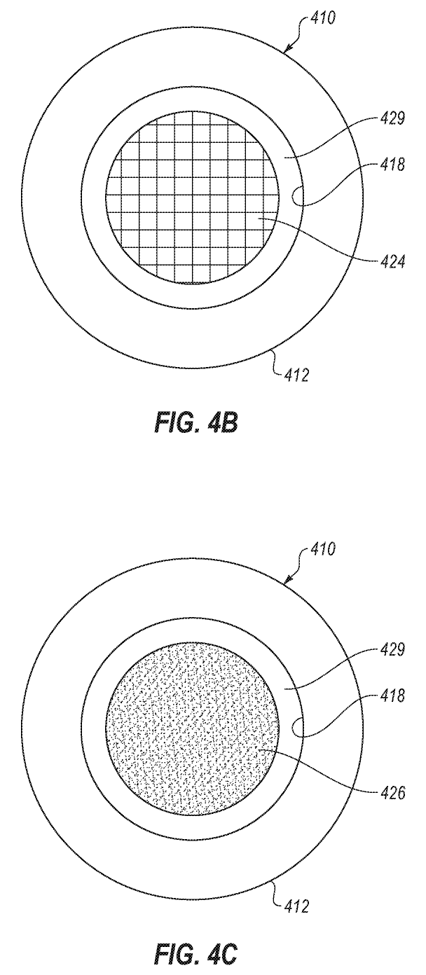

As illustrated in FIG. 4A, in one embodiment an expandable docking station/device/valve adapter/landing zone/prestent 410 (hereinafter “docking station”) includes one or more sealing portions 412, a valve seat 418, and one or more retaining portions 414. The sealing portion(s) 412 provide a seal between the docking station 410 and an interior surface of the circulatory system. The valve seat 418 provides a supporting surface for implanting or deploying a valve 429 in the docking station 410 after the docking station 410 is implanted in the circulatory system. Optionally, the docking station 410 and the valve 429 may be integrally formed, for example, in one embodiment, the valve seat 418 may be omitted or the valve 429 may be formed into or coupled to the valve seat 418 prior to deploying the docking station 410. When integrally formed, the docking station 410 and the valve 429 may be deployed as a single device, rather than first deploying the docking station 410 and then deploying the valve 429 into the docking station. Any of the docking stations and/or valve seats 418 described herein may be provided or formed with an integrated valve 429. In another embodiment, the docking station 410 accommodates standardized sizes of heart valves, e.g., conforms to various aortic sizes and provides a consistently sized valve seat 418 into which artificial valves may be deployed.

In some embodiments, the docking station 410 may include a retaining portion that may help retain the docking station 410 and the valve 429 at the implantation position or deployment site in the circulatory system. The retaining portion may take a wide variety of different forms. In one example embodiment, the retaining portion includes friction enhancing features that reduce or eliminate migration of the docking station 410. The friction enhancing features may take a wide variety of different forms. For example, the friction enhancing features may comprise barbs, spikes, texturing, adhesive, and/or a cloth or polymer cover with high friction properties on the retaining portions. Such friction enhancing features may also be used on any of the various docking stations or retaining portions described herein.

Expandable docking station 410 and valve 429 as described in the various embodiments herein are also representative of a variety of docking stations and/or valves described herein or that might be known or developed, e.g., a variety of different types of valves could be substituted for and/or used as valve 429 in the various docking stations.

FIGS. 4A-4C illustrate a representative example of the operation of the docking stations 410 and valves 429 disclosed herein. In the example of FIGS. 4A-4C, the docking station 410 and valve 429 are deployed in the aortic valve (AV). However, the docking station 410 and valve 429 may be deployed in any interior surface within the heart or a lumen of the body. For example, the various docking stations and valves described herein may be deployed in the inferior vena cava (IVC), the superior vena cava (SVC), the tricuspid valve (TV), the pulmonary valve (PV), pulmonary artery, the mitral valve (MV), aorta, or other vasculature/lumens in the body.

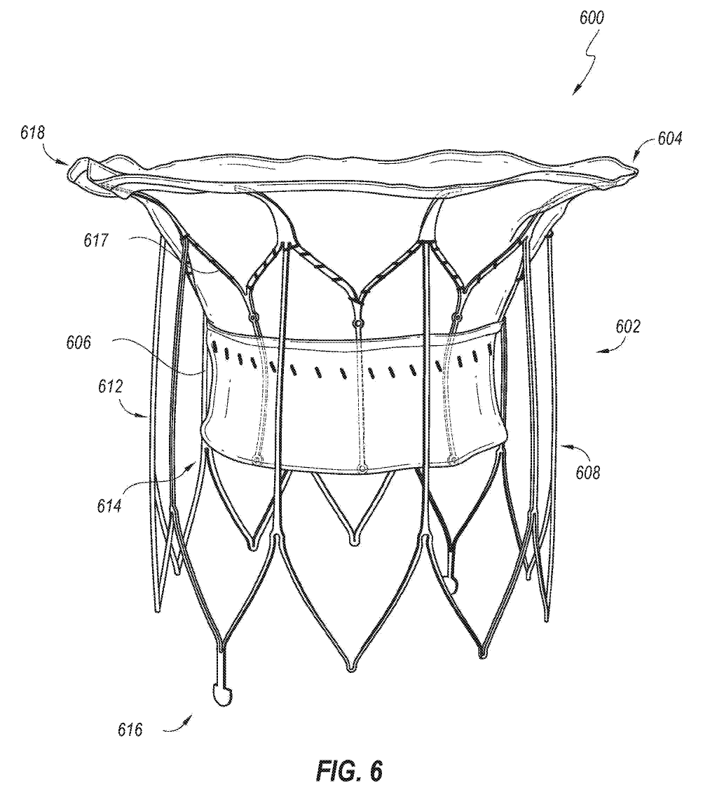

FIG. 4B illustrates space 424 that represents the valve 429 being open when the heart is in the diastolic phase. A variety of types of valves may be used that may open and close in a variety of ways (e.g., including valves with leaflets of tissue that open then coapt to close), so the drawings are meant to be representative of a variety of valves that may operate in different ways. FIG. 6B does not show the interface between the docking station 410 and the aortic valve to simplify the drawing. The cross-hatching in FIG. 6B represents blood flow through the valve 429. In an example embodiment, blood is prevented or inhibited from flowing between the aortic valve and the docking station 410 by the sealing portion 412 and blood is prevented or inhibited from flowing between the docking station 410 and the valve by implanting or seating the valve in the seat 418 of the docking station 410. In this example, blood only substantially flows or is only able to flow through the valve 429 when the valve is open (e.g., in certain embodiments, only when the heart is in the diastolic phase). FIG. 4C illustrates the valve 429 and docking station 410, when the valve 429 is closed (e.g., when implanted in the AV and the heart H is in the systolic phase). When implanted in the AV and the heart is in the systolic phase, the valve 429 closes. FIG. 4C is meant to be representative of a variety of valves, even though those valves may close in different ways.

In one example embodiment, the docking station 410 acts as an isolator that prevents or substantially prevents radial outward forces of the valve 429 from being transferred to the inner surface of the circulatory system. In another example embodiment, the docking station 410 is also designed to accommodate and conform to various aortic sizes, thereby allowing accommodation of standardized sizing of heart valves. In one embodiment, the docking station 410 includes a valve seat 418 that resists expansion, e.g., is not expanded radially outwardly (e.g., the diameter of the valve seat does not increase) or is not substantially expanded radially outward (e.g., the diameter of the valve seat increases by less than 4 mm) by the radially outward force of the transcatheter valve or valve 429. The valve seat may be configured such that expansion of a THV/valve 429 increases the diameter of the valve seat only to a diameter less than an outer diameter of the docking station 410 when the docking station is implanted. Retaining portions and sealing portions 412 may be configured to impart only relatively small radially outward forces on the inner surface of the circulatory system (as compared to the radially outward force applied to the valve seat 418 by the valve 429). Having a valve seat 418 that is stiffer or less radially expansive than the outer portions of the docking station (e.g., retaining portions and sealing portions 412), as in the various docking stations described herein, provides many benefits, including allowing a THV/valve 429 to be implanted in vasculature or tissue of varying strengths, sizes, and/or shapes. The outer portions of the docking station may better conform to the anatomy (e.g., vasculature, tissue, heart, etc.) without putting too much pressure on the anatomy, while the THV/valve 29 may be firmly and securely implanted in the valve seat 418 with forces that will prevent or mitigate the risk of migration or slipping.

The docking station 410 may include any combination of one or more than one different types of valve seats 418, retaining portions 414, and/or sealing portions 412. For example, the valve seat 418 may be a separate component that is attached to the frame of the docking station 410, while the sealing portion is integrally formed with the frame of the docking station. Also, the valve seat 418 may be a separate component that is attached to the frame of the docking station 410, while the sealing portion 412 is a separate component that is also attached to the frame of the docking station. Optionally, the valve seat 418 may be integrally formed with the frame of the docking station 410, while the sealing portion is integrally formed with the frame of the docking station. Further, the valve seat 418 may be integrally formed with the frame of the docking station 410, while the sealing portion is a separate component that is attached to the frame of the docking station 410. The sealing portion 412, the valve seat 418, and one or more retaining portions 414 of the various docking stations herein may take a variety of different forms and characteristics.

The docking station 410 may be made from a highly flexible metal, metal alloy, or polymer. Examples of metals and metal alloys that may be used include, but are not limited to, nitinol and other shape memory alloys, elgiloy, and stainless steel, but other metals and highly resilient or compliant non-metal materials may be used to make the docking station 410 . These materials may allow the frame to be compressed to a small size, and then when the compression force is released, the frame will self-expand back to its pre-compressed diameter and/or the frame may be expanded by inflation of a device positioned inside the frame. The docking station 410 may also be made of other materials and be expandable and collapsible in different ways, e.g., mechanically-expandable, balloon-expandable, self-expandable, or a combination of these.

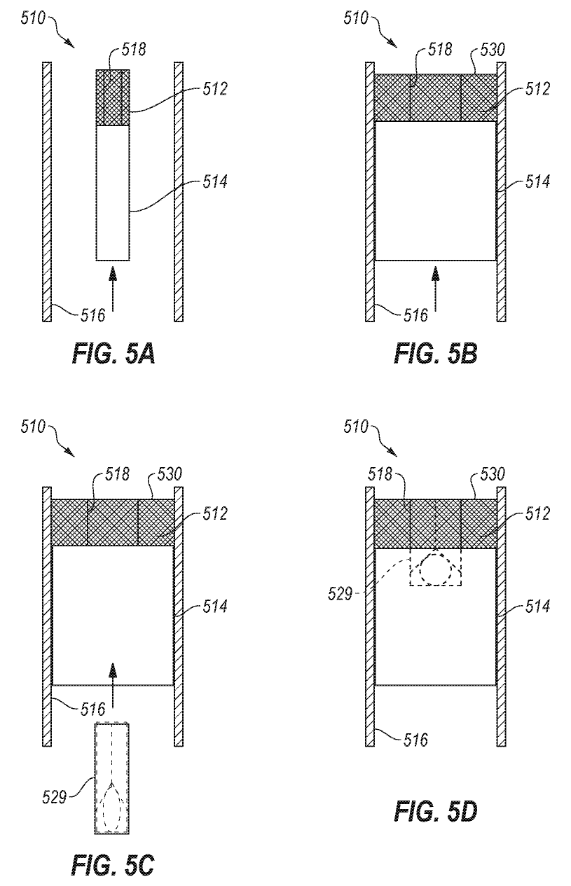

FIGS. 5A-5D schematically illustrate an example deployment of the docking station 510 and valve 529 in the circulatory system, or other docking station, e.g., as described in FIGS. 7-9. Referring to FIG. 5A, the docking station 510 is in a compressed form/configuration and is introduced to a deployment site in the circulatory system. For example, the docking station 510, may be positioned at a deployment site in a SVC, IVC, aorta, or other location. Referring to FIG. 5B, the docking station 510 is expanded in the circulatory system such that the sealing portion(s) 512 and/or the retaining portion(s) 514 engage the inside surface 516 of a portion of the circulatory system. The docking station may be self-expanding, and may be advanced from a delivery capsule into the expanded state, or plastically expandable such that it may be expanded using a balloon or other expansion device. Referring to FIG. 5C, after the docking station 510 is deployed, the valve 529 is in a compressed form and is introduced into the valve seat 518 of the docking station 510. Referring to FIG. 5D, the valve 529 is expanded in the docking station, such that the valve 529 engages the valve seat 518 and the seat 518 of the docking station 510 supports the valve. The docking station 510 allows the valve 529 to operate within the expansion diameter range for which it is designed. In the examples depicted herein, the docking station 510 is longer than the valve. However, in some embodiments the docking station 510 may be the same length or shorter than the length of the valve 529. Similarly, the valve seat 518 may be longer, shorter, or the same length as the length of the valve 529. Any of the docking station embodiments described herein may be deployed in the manner described above.

### Second Representative Embodiment

FIG. 6 illustrates a side view of the components of another example docking station 600 configured to dock and/or support one or more prosthetic valves and/or valve components in accordance with one or more embodiments of the present disclosure. The docking station 600 may comprise a frame 602 and/or a sealing element 604, which may include a skirt, covering, and/or similar device. The frame 602 may be configured to form an inner frame 606 and/or an outer frame 608. The inner frame 606 may form a first diameter that is less than a second diameter of the outer frame 608. The inner frame 606 and outer frame 608 may be extensions of a common device and/or may extend from each other.

The frame 602 may comprise a wire frame formed by a network of struts 612 (e.g., wires, cords, and/or bars) forming one or more cells 614. The frame 602 may mostly comprise empty space and/or cells 614 between the struts 612. For example, the struts 612 may be generally thin and/or may be spaced apart to create relatively large gaps between the struts 612, as shown in FIG. 6. In some examples, the outer frame 608 may comprise a series of longitudinally extending struts 612 aligned in series and/or in parallel around a circumference of the docking station 600. Each of the struts 612 may join to one or more adjacent struts 612 at a proximal end 616 of the docking station 600 and/or may join with one or more adjacent struts 612 and/or with the inner frame 606 at a distal end 618 of the docking station 600.

The inner frame 606 may similarly comprise a network of generally thin, elongate, and/or spaced apart struts 612. In some examples, one or more struts 612 of the inner frame 606 may extend generally in series and/or in parallel longitudinally along a length of the docking station 600. The one or more struts 612 of the inner frame 606 may be disposed between struts 612 of the outer frame 608.

In some examples, the outer frame 608 may have a generally cylindrical form and/or may at least partially enclose a complete circumference of the inner frame 606 and/or of a portion of the sealing element 604. The outer frame 608 may comprise a network of struts 612, which may include wires, arms, bars, cords, walls, and/or similar components forming one or more cells 614 and/or openings through the outer frame 608. The one or more cells 614 may be configured to allow blood flow through the outer frame 608. The cells 614 may have any suitable shape and/or size. The outer frame 608 may form generally elongate cells 614 extending approximately an entire length of the outer frame 608 and/or from a proximal end 616 to a distal end 618 of the docking station 600. The one or more cells 614 may have triangular forms at end points of the one or more cells 614 (or stated another way, V-shaped junctions). However, the one or more struts 612 may form cells 614 having different shapes. For example, the struts 612 may be configured to form generally rectangular, oval, curved (e.g., U-shaped junctions), and/or diamond-shaped cells 614. The struts 612 may include any structure, including wire-like and/or generally thin forms, or thicker metal bars, rods, or spans. In some examples, the inner frame 606 and/or outer frame 608 may be configured to maintain a uniform structure and/or strut 612 pattern along a length of the frame 602.The docking station 600 may be configured for delivery and/or placement at an aortic valve of the heart.

At a distal end 618 of the docking station 600, the outer frame 608 and inner frame 606 may join together and/or the inner frame 606 may extend away from the outer frame 608 and/or along an inner lumen formed by the outer frame 608. In some examples, the outer frame 608 and inner frame 606 may both have a generally flared and/or conical form at or near the distal end 618 of the docking station 600. The inner frame 606 may have a flaring angle that is greater than the outer frame 608 such that the diameter of the inner frame 606 may be less than the diameter of the outer frame 608. The inner frame 606 may be configured to extend along at least a portion of the length of the outer frame 608.

The flared end (e.g., distal end 618) of the outer frame 608 and/or inner frame 606 may be configured to engage an atrium and/or other chamber when implanted. The sealing element 604 (e.g., a sealing skirt) may be configured to extend along the inner frame 606 and/or may be configured to wrap around the outer frame 608 and/or extend between the outer frame 608 and the native tissue for a short distance. The outer frame 608 may be configured to extend along and/or against the aortic wall and/or other blood vessel. The sealing element 604 may be configured to extend from the outer frame 608 to an inner surface of the inner frame 606. The sealing element 604 may be configured for engagement with a prosthetic valve and/or other implant. For example, the sealing element 604 may be configured to provide a mounting surface for a prosthetic valve and/or may be configured to securely hold the prosthetic valve. The sealing element 604 may be configured to extend along only a portion of the docking station 600 such that the sealing element 604 may not extend across one or more branching vessels of the blood vessel. For example, the sealing element 604 may extend from an implanted valve (not shown) along the inner frame 606 and to a junction between the inner frame 606 and the outer frame 608.

In some examples, the outer frame 608 may be formed as or include one or more outward bulbs configured to extend outwardly to facilitate anchoring of the docking station 600 within a blood vessel. The frame 602 may comprise one or more downward-extending arms 617 (e.g., extending towards the proximal end 616) configured to form the inner frame 606. For example, the arms 617 may extend downwardly from the outer frame 608 at or near the distal end 618 of the frame 602. The one or more arms 617 may be configured to extend at an acute angle away from the outer frame into the lumen of the frame 602 and/or may extend generally in parallel with the outer frame 608 at or near distal ends of the one or more arms 617.

### Third Representative Embodiment

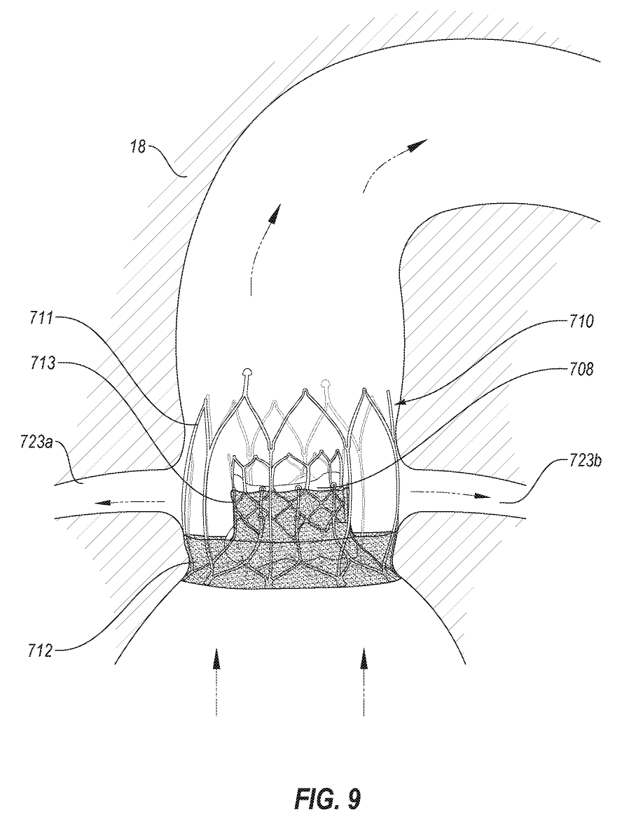

FIG. 7 illustrates a side view of a docking station 710 having a self-expandable outer frame 711 and an inner frame 713. FIG. 8 illustrates a top view of the docking station 710 of FIG. 7. FIG. 9 depicts the docking station 710 implanted in the aortic arch of the heart. FIG. 9 also depicts a sealing skirt 712 which is not shown in FIGS. 7 and 8 for convenience.

Referring to FIG. 7, the docking station 710 may include an inner wire frame 713 (“inner frame”) and an outer wire frame 711 (“outer frame”) concentrically joined at an upstream juncture 714. The outer frame 711 may include an open mesh which may include a series of cells 715 around the circumference of the outer frame 711, each single cell 715 may extend some or all of the length 718 of the outer frame 711. The inner frame 713 may be joined to the outer frame 711 at the apices 716 of the cells 715 of the outer frame 711 and extend toward the inside of the outer frame 711. In some embodiments, the inner frame 713 may include single wires extending inside the outer frame 711 from the apex 716 to a region 717 inside the outer frame 711. In these and other embodiments, the inner frame 713 may extend along a same length or less as the outer frame 711. Stated another way, the inner frame 713 may not extend past the downstream end of the outer frame 711. In some embodiments, the inner frame 713 may include connections between the struts of the inner frame 713. For example, a reinforcing strut or other material may connect the inner frame 713. The cross section of the outer frame 711/inner frame 713 junction 714 may be “V-shaped” or “U-shaped.”

In some embodiments, the apices 716 may be rounded, bulged, bulbous, or any other shape which may reduce or minimize trauma caused by the apices 71 when interfacing with the anatomy of the patient. While such apices may not be completely atraumatic, such an embodiment may reduce the amount of damage or penetration into the tissue.

In some embodiments, a sealing skirt 712 may be attached to a proximal/upstream end of the docking station 710. For example, the sealing skirt 712 may be coupled to the docking station 710 at the joined outer frame 711/inner frame 713 and may extend from the inside end of the inner frame 713 (e.g., the valve seat 720) to the apices 716, and then turn upward along the outside of the outer frame 711. In some embodiments, the distance the sealing skirt 712 covers the outer frame 711 may extend a predetermined distance as desired by the user or clinician, and may typically include a short distance. For example, the distance may be selected to avoid sealing against the aortic ostia 723a and 723b as illustrated in FIG. 9, and in this manner, ostium blockage may be eliminated or reduced. For example, the distance may include 0.5 mm, 1 mm, 2 mm, 3 mm, 4 mm, 5 mm, or any range bounded by any of the foregoing values. In some embodiments, the distance may be one tenth of the length 718 of the docking station 710, one fifth of the length 718, one fourth of the length 718, one third of the length 718, or any other distance, or any range bounded by any of the foregoing values.

In some embodiments, the docking station 710 may be wider (e.g., have a larger diameter) than the length 718 of the docking station 710. In some embodiments, the relative dimensions may be described as the width relative to the length 718. For example, in some embodiments, a ratio of the width to the length 718 may include 1:1, 1.25:1, 1.5:1, 1.75:1, 2:1, 2.5:1, 3:1, 4:1, or any other ratio bounded by any of the foregoing values, such as between 1.25:1 and 1.75:1.

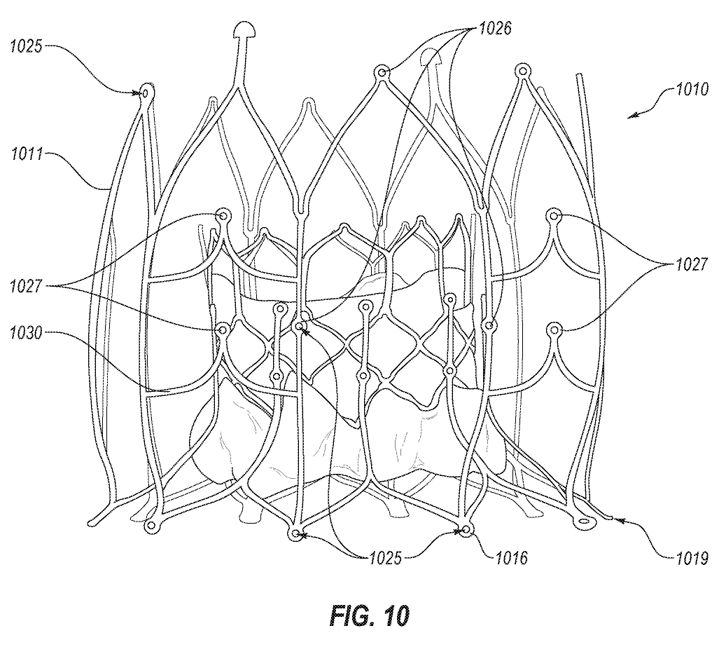

In some embodiments, the docking station 710 may be crimpable and may be self-expanding. For example, the docking station 710 may be crimped or otherwise compressed to be loaded onto a delivery catheter. When deploying the docking station 710, the docking station 710 may expand from its compressed state to an expanded state (e.g., self-expansion when made of nitinol or other shape-memory alloy). Additionally or alternatively, the docking station 710 may be balloon-expandable. In some embodiments, the inner frame 713 and/or the outer frame 711 of the docking station 710 may include a single cell that spans along the length 718 of the docking station 710, which may or may not be repeated about the entire circumference of the docking station 710. Additionally or alternatively, the inner frame 713 and/or the outer frame 711 may include multiple cells along the length 718 of the docking station 710. In some embodiments, cells of the inner frame 713 and/or the cells 715 of the outer frame 711 may include reinforcing components (e.g., struts, bridges, wires, etc., as explained with reference to FIG. 10) to strengthen one or more of the cells. In some embodiments, the reinforcing components may divide a given cell into multiple cells. In these and other embodiments, less than all of the cells may include the reinforcing components. For example, every other cell, every third cell, or any other configuration of cells may include the reinforcing components. An example of such a configuration is illustrated in FIG. 10.

In some embodiments, the length 718 of the docking station 710 may be variable. For example, a set or series of lengths may be manufactured or designed such that a clinician may select an appropriately sized docking station 710 based on the anatomy of a given patient. In some embodiments, the length may be selected based upon a length that a clinician determines would facilitate anchoring or adequately support anchoring. Additionally or alternatively, the length may be selected based on a size and/or location of coronary ostium. For example, the length may be selected such that there may be a space within the inner frame 713 to hold at least a section of a prosthetic valve (e.g., prosthetic valve 708 in FIG. 9) while leaving a gap 721 between the inner frame 713 and the outer frame 711 to allow for adequate blood flow into the coronary ostium.

In some embodiments, the gap 721 between the outer frame 711 and the inner frame 713 may be sized to provide adequate blood flow into the coronary ostium and/or to permit subsequent procedures to be performed associated with the coronary ostium without disturbing the deployed docking station 710. For example, the gap 721 may be sized to allow a coronary procedure catheter to be inserted, to allow for the performance of coronary procedures without, for example, moving, removing, or otherwise disturbing the docking station 710. For example, a coronary ablation may be performed, all or portions of a coronary bypass may be performed, or any other procedure involving the ostium may be performed.

In some embodiments, the docking station 710 may include a sealing skirt 712 that extends from the distal tip 719 of the docking station 710, rather than covering a portion along the outer frame 711. In these and other embodiments, the sealing skirt 712 follows and traverses the inner frame 713 and creates a seal from the distal tip 719 of the docking station 710 against the anatomy of a patient. Such an embodiment may permit greater access to the ostia, which is beneficial for certain aortic procedures, while posing a potential increase in risk of failing to create an adequate seal against the anatomy.

Because there is a narrow, shorter landing zone when implanting to replace the aortic valve as compared to docking stations deployed in other locations, such as the interior vena cava, the sealing skirt 712 may be designed to include a majority of the material of the sealing skirt 712 along the inner frame 713 rather than the outer frame 711, which may keep the outer regions of the docking station 710 accessible. The docking station 710 with the sealing skirt 712 may allow for both improved and facilitated anchoring of a prosthetic valve, and improved accessibility for coronary intervention.

In some embodiments, the docking station 710 may include a flange 725. The flange 725 may be used to create a better seal against the anatomy of a patient. For example, the flange 725 proximate the distal tip 719 may contribute to creating a seal against the surrounding anatomy as well as keeping the docking station 710 in its place. In another embodiment, the flange 725 proximate the distal tip 719 is used to create the proper desired seal.

In some embodiments, a foam lining (not shown) may be associated with the flange 725. For example, between the apices 716 and the sealing skirt 712 the foam lining may provide protection for the docking station 710 and/or protection for the native anatomy of the patient. Additionally or alternatively, the foam lining may facilitate forming a seal between the docking station 710 and the anatomy of the patient.

In some embodiments, the docking station 710 may vary in length to change the anchoring strength of the docking station 710. For example, the docking station 710 may extend further along the aorta to facilitate anchoring of the docking station 710 into place. In some embodiments, the longer length may be accomplished with additional cells and/or with longer individual cells.

As illustrated in FIG. 9, the docking station 710 may include a prosthetic valve 708 deployed within the docking station 710 when the docking station is deployed to replace an aortic valve. The docking station 710 may seat the prosthetic valve 708 to reduce backflow or regurgitation while also reducing or eliminating blockage of coronary ostia 723a and/or 723b.

In some embodiments, the docking station 710 may include one or more radiopaque markers 726 (such as the radiopaque markers 726a-c) that may facilitate understanding the position of the docking station 710 relative to a valve to be disposed within the docking station 710. For example, the radiopaque markers 726 may facilitate guidance, orientation, and otherwise facilitate a procedure of guiding the valve through the body of the patient into the docking station 710, and/or otherwise positioning or verifying the position of the docking station 710 when seating the valve into the docking station. For example, the radiopaque markers 726 may facilitate a view into an axial or radial orientation, state of deployment, or other information for the clinician when performing a procedure involving the docking station 710 and/or the valve. The radiopaque markers 726 may be made of any radiopaque material, such as tantalum, bismuth, iodine, barium, or gold.

FIG. 10 illustrates a docking station 1010 having radiopaque markers 1025 and/or horizontal reinforcing struts 1030, in accordance with one or more embodiments of the present disclosure. The docking station 1010 may be similar or comparable to the docking station 410, 510, 600, and/or 710.

The radiopaque markers 1025 may facilitate understanding the position of the docking station 1010 during a procedure. For example, the docking station 1010 may include one or more radiopaque markers 1025 (such as a first set of radiopaque markers 1026 and/or a second set of radiopaque markers 1027) to facilitate guidance, orientation, and otherwise facilitate a procedure of guiding the docking station 1010 through the body of the patient to the target or deployment location, deploying the docking station 1010, and/or otherwise positioning or verifying the position of the docking station 1010 in the desired position. For example, the radiopaque markers 1025 may facilitate a view into an axial or radial orientation, state of deployment, or other information for the clinician when performing a procedure involving the docking station 1010. The radiopaque markers 1025 may be made of any radiopaque material, such as tantalum, bismuth, iodine, barium, or gold.

In some embodiments, the first series of radiopaque markers 1026 may be positioned so as to coincide with a first cell that is open. For example, the first series of radiopaque markers 1026 may be disposed on the outer frame 1011 at or near the position of the first open cell, e.g., by being coined into an opening or gap in the outer frame 1011. In these and other embodiments, the first series of radiopaque markers 1026 may be disposed at any number or variation of positions about the first cell to facilitate visual identification of the first cell as an open cell. By doing so, the clinician may be able to orient the docking station 1010 so that the first cell is positioned against the coronary ostia.

In some embodiments, the second series of radiopaque markers 1027 may be positioned on one or more of the reinforcing struts 1030 to facilitate identification of cells which are not open cells, or in other words, identifying the cells that have reinforcing struts 1030 creating potential blockage across the cells. For example, by positioning the second series of radiopaque markers 1027 on the reinforcing struts 1030, a clinician may orient the docking station 1010 so that the reinforcing struts 1030 are positioned away from coronary ostia.

In some embodiments, the docking station 1010 may include only the first set of radiopaque markers 1026, only the second set of radiopaque markers 1027, or both the first and the second set of radiopaque markers 1026/1027.

In these and other embodiments, the first radiopaque markers may be disposed at generally consistent positions about the docking station 1010. For example, the first set of radiopaque markers 1026 may be disposed periodically around the circumference of each of the cells that are open cells. As another example, the second set of radiopaque markers 1027 may include one or more radiopaque markers disposed on each of the reinforcing struts 1030 at a regular and/or consistent position. While a given quantity of radiopaque markers are illustrated in each of the first and the second set of radiopaque markers 1026/1027, it will be appreciated that any quantity is contemplated, such as three, four, six, eight, ten, twelve, fifteen, twenty, or any range bounded by any of the preceding values, such as between three and fifteen.

In some embodiments, the outer frame 1011 may include one or more reinforcing struts 1030 to provide greater stability and/or rigidity to the docking station 1010. For example, the reinforcing struts 1030 may facilitate the docking station 1010 withstanding the force with which the blood flows through the aortic valve. Additionally or alternatively, the reinforcing struts 1030 may facilitate the docking station 1010 retaining its expanded state. While illustrated as reinforcing across the cells of the outer frame 1011, it will be appreciated that other portions of the docking station 1010 may also be reinforced such as the inner frame, the valve seat, the flange, or any other portions or regions of the docking station 1010.

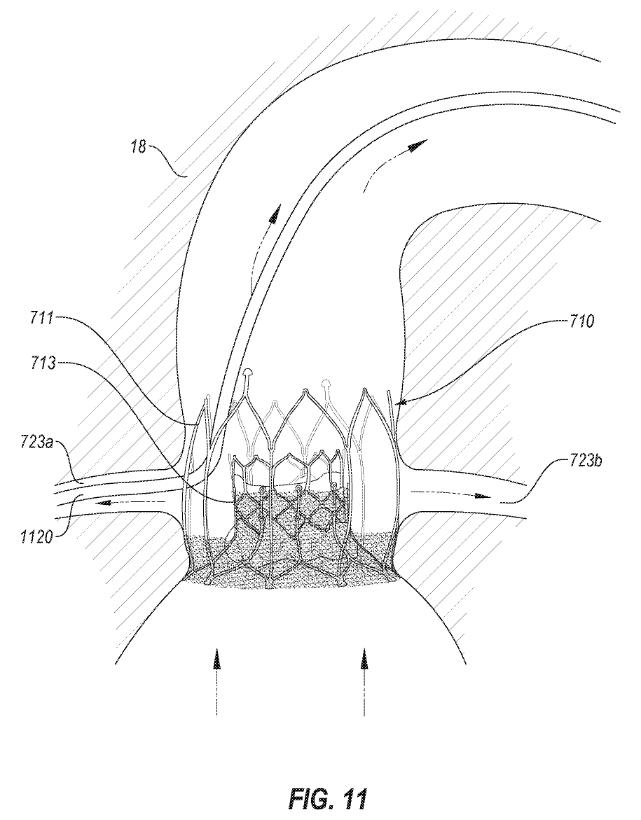

FIG. 11 illustrates a procedure catheter 1120 for performing a procedure in a coronary artery of a patient after the patient has already received the docking station 710 and/or a replacement aortic valve 708.

As illustrated in FIG. 11, a patient may have previously received a procedure in which their aortic valve was replaced using the docking station 710. After receiving the docking station 710, it may be determined by a clinician that another procedure would be advisable for the patient that involves the coronary ostia 723a/732b.

In one example procedure, blood flow to the heart of the patient may be slowed, restricted, or stopped by blockage in the coronary arteries. In these and other embodiments, a stent may be moved in place of the blockage and expanded (e.g., by a balloon) to open up the blockage and allow the blood to flow more regularly through the coronary artery (e.g., an angioplasty such as a balloon angioplasty). Additionally or alternatively, any other procedures which may access the coronary arteries may be performed without disturbing the docking station 710 and/or the valve 708.

In these and other embodiments, the clinician may guide the procedure catheter 1120 through the anatomy of the patient, over the aortic arch 18, and down through the gap between the outer frame 711 and the inner frame 713 of the docking station 710. The procedure catheter 1120 may then pass through the docking station 710 past the outer frame 711 and into the coronary ostia 723a/732b. In these and other embodiments, by preventing blockage of the coronary ostia 723a/723b, the blood flow may be maintained and a clinician may have access to the coronary ostia 723a/723b should another treatment or procedure be advisable.

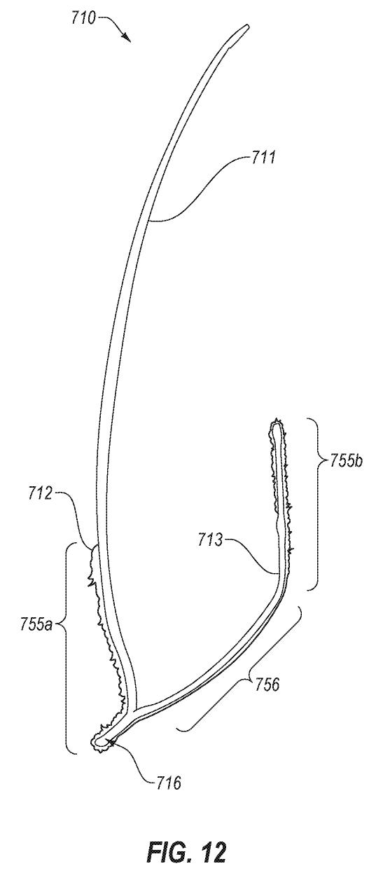

FIG. 12 illustrates a partial section view of the docking station 710 of FIG. 7. As illustrated in FIG. 12, the docking station 710 may include the outer frame 711 and the inner frame 713 with the apex 716 between them and the sealing skirt 712 on the docking station 710.

As illustrated in FIG. 12, in some embodiments, the sealing skirt 712 may include portions of varying thickness. For example, the sealing skirt 712 may include a first portion 755a with an increased thickness. The increased thickness may include a thicker nap, depth, or other variation in the material forming the sealing skirt. The first portion 755a with the increased thickness may facilitate the formation of the seal between the outer frame 711 and the anatomy of the patient in the first portion 755a. Additionally or alternatively, the sealing skirt 712 may include a second portion 755b with an increased thickness. The second portion 755b with the increased thickness may facilitate the formation of the seal between the inner frame 713 and the artificial valve.

In some embodiments, the sealing skirt 712 may include a third portion 756 with a thinner depth relative to the first and second portions 755a-b. The thinner portion of the sealing skirt 712 may result in a decreased absorption of blood, decreased clot formation, decreased foreign body response, and/or the reduction or minimization of any negative effects of the material of the sealing skirt 712 while still enjoying the benefit of the increased thickness for improved sealing in the first and second portions 755a-b.

In some embodiments, one or more sutures may be used to suture, stitch, sew, or otherwise attach the sealing skirt 712 to the outer frame 711 and/or the inner frame 713.

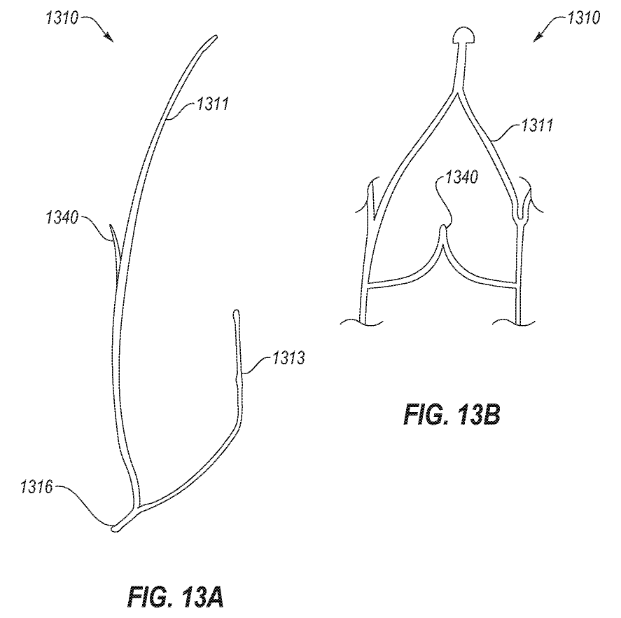

FIG. 13A illustrates a partial section view of a docking station 1310 including a retaining barb 1340. FIG. 13B illustrates a front view of a portion of the docking station 1310 of FIG. 13A. The docking station 1310 may be similar or comparable to the docking station 710 of FIGS. 7-12. For example, the docking station 1310 may include an outer frame 1311 that may be similar or comparable to the outer frame 711, an inner frame 1313 that may be similar or comparable to the inner frame 1313, and one or more apices 1316 that may be similar or comparable to the apices 716. While other features and portions are not illustrated, it will be appreciated that any of the features, benefits or other aspects of the docking station 710 may be included with and/or be applicable to the docking station 1310.

As illustrated in FIGS. 13A-13B, the docking station 1310 may include a retaining barb 1340 that may be configured to penetrate or otherwise interface with the tissue of a patient to facilitate maintaining the docking station 1310 in position even when the heart is pumping. For example, the retaining barb 1340 may be configured to bite into the tissue to lock the docking station 1310 into place within the aortic valve. Additionally or alternatively, the retaining barb 1340 may be positioned to bite into the wall of the aorta leading up to the aortic arch past the aortic ostia.

In some embodiments, the retaining barb 1340 may include a flexing portion that may be disposed between two struts of the outer frame 1311 that may flex outwards away from the radial center of the docking station 1310 when expanding from a compressed state to an expanded state.

While illustrated as a single barb in a single location, it will be appreciated that the retaining barb 1340 may be located at any position along the length of the outer frame 1311. Additionally or alternatively any number of retaining barbs 1340 may be included, such as two, three, four, five, or ten.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A docking station for use in aortic valve replacement, comprising: an inner wire frame forming a valve seat configured to receive or couple to an artificial valve; an outer wire frame joined to the inner wire frame at one or more apices, the outer wire frame comprising a series of cells about a circumference of the outer wire frame; and a sealing skirt that extends from the valve seat to the apices and turns upward along an exterior region of the outer wire frame, wherein a ratio of a width to a length of the docking station includes a ratio greater than 1:1 and less than 3:1.

Example 2. Any of the foregoing examples, such as example 1, wherein the sealing skirt turns upward less than 5 mm.

Example 3. Any of the foregoing examples, such as example 2, wherein a distance the sealing skirt turns upward prevents sealing against one or more aortic ostia.

Example 4. Any of the foregoing examples, such as any of examples 1-3, wherein the inner wire frame extends from the apices no further than a downstream end of the outer wire frame.

Example 5. Any of the foregoing examples, such as any of examples 1-4, wherein the inner wire frame is formed of one or more single wires extending from the apices to the valve seat.

Example 6. Any of the foregoing examples, such as any of examples 1-5, wherein a junction between the outer wire frame and the inner wire frame forms a V-shaped cross-section.

Example 7. Any of the foregoing examples, such as any of examples 1-5, wherein a junction between the outer wire frame and the inner wire frame forms a U-shaped cross-section.

Example 8. Any of the foregoing examples, such as any of examples 1-7, wherein the docking station is crimpable.

Example 9. Any of the foregoing examples, such as any of examples 1-8, wherein the docking station is self-expanding.

Example 10. Any of the foregoing examples, such as any of examples 1-8, wherein the docking station is balloon-expandable.

Example 11. Any of the foregoing examples, such as any of examples 1-10, wherein an interior region of the inner wire frame conforms to one or more sizes of one or more prosthetic heart valves.

Example 12. Any of the foregoing examples, such as any of examples 1-11, wherein the docking station further comprises a foam lining underneath the sealing skirt.

Example 13. Any of the foregoing examples, such as example 12, wherein the foam lining covers at least the apices.

Example 14. Any of the foregoing examples, such as any of examples 1-13, wherein at least one of the cells of the outer wire frame is spaced to permit a secondary catheter device to traverse a gap between the outer wire frame and the inner wire frame in a deployed state of the docking station to access aortic ostia by the secondary catheter device by passing through the gap between the inner wire frame and the outer wire frame and into the aortic ostia.

Example 15. Any of the foregoing examples, such as any of examples 1-14, wherein a first cell of the series of cells of the outer wire frame includes a first reinforcing strut within the first cell, and a second cell of the series of cells of the outer wire frame is defined by a perimeter of the second cell without a second reinforcing strut.

Example 16. Any of the foregoing examples, such as example 15, further comprising one or more radiopaque markers associated with the second cell.

Example 17. Any of the foregoing examples, such as any of examples 15-16, wherein the radiopaque markers are positioned about the second cell.

Example 18. Any of the foregoing examples, such as any of examples 15-17, wherein at least one of the radiopaque markers is positioned on the first reinforcing strut.

Example 19. Any of the foregoing examples, such as any of examples 1-18, wherein the ratio of the width to the length includes between 1:1 and 2:1.

Example 20. Any of the foregoing examples, such as any of examples 1-19, wherein the ratio of the width to the length includes between 1.25:1 and 1.75:1.

Example 21. Any of the foregoing examples, such as any of examples 1-20, further comprising the artificial valve formed into the valve seat.

Example 22. Any of the foregoing examples, such as any of examples 1-22, further comprising the artificial valve coupled to the valve seat prior to implantation of the docking station.

Example 23. A method for reducing ostium blockage, the method comprising implanting a docking station, the docking station comprising: an inner wire frame forming a valve seat configured to receive or couple to an artificial valve; an outer wire frame joined to the inner wire frame at one or more apices, the outer wire frame comprising a series of cells about a circumference of the outer wire frame; and a sealing skirt that extends from the valve seat to the apices and turns upward along an exterior region of the outer wire frame, wherein a ratio of a width to a length of the docking station includes a ratio greater than 1:1 and less than 3:1.

Example 24. Any of the foregoing examples, such as example 23, wherein the sealing skirt turns upward less than 5 mm.

Example 25. Any of the foregoing examples, such as example 24, wherein a distance the sealing skirt turns upward prevents sealing against one or more aortic ostia.

Example 26. Any of the foregoing examples, such as any of examples 23-25, wherein the inner wire frame extends from the apices no further than a downstream end of the outer wire frame.

Example 27. Any of the foregoing examples, such as any of examples 23-26, wherein the inner wire frame is formed of one or more single wires extending from the apices to the valve seat.

Example 28. Any of the foregoing examples, such as any of examples 23-27, wherein a junction between the outer wire frame and the inner wire frame forms a V-shaped cross-section.

Example 29. Any of the foregoing examples, such as any of examples 23-27, wherein a junction between the outer wire frame and the inner wire frame forms a U-shaped cross-section.

Example 30. Any of the foregoing examples, such as any of examples 23-29, wherein the docking station is crimpable.

Example 31. Any of the foregoing examples, such as any of examples 23-30, wherein the docking station is self-expanding.

Example 32. Any of the foregoing examples, such as any of examples 23-30, wherein the docking station is balloon-expandable.

Example 33. Any of the foregoing examples, such as any of examples 23-32, wherein an interior region of the inner wire frame conforms to one or more sizes of one or more prosthetic heart valves.

Example 34. Any of the foregoing examples, such as any of examples 23-33, wherein the docking station further comprises a foam lining underneath the sealing skirt.

Example 35. Any of the foregoing examples, such as example 34, wherein the foam lining covers at least the apices.

Example 36. Any of the foregoing examples, such as any of examples 23-35, wherein at least one of the cells of the outer wire frame is spaced to permit a secondary catheter device to traverse a gap between the outer wire frame and the inner wire frame in a deployed state of the docking station to access aortic ostia by the secondary catheter device by passing through the gap between the inner wire frame and the outer wire frame and into the aortic ostia.

Example 37. Any of the foregoing examples, such as any of examples 23-36, wherein a first cell of the series of cells of the outer wire frame includes a first reinforcing strut within the first cell, and a second cell of the series of cells of the outer wire frame is defined by a perimeter of the second cell without a second reinforcing strut.

Example 38. Any of the foregoing examples, such as example 37, further comprising one or more radiopaque markers associated with the second cell.

Example 39. Any of the foregoing examples, such as any of examples 37-38, wherein the radiopaque markers are positioned about the second cell.

Example 40. Any of the foregoing examples, such as any of examples 37-39, wherein at least one of the radiopaque markers is positioned on the first reinforcing strut.

Example 41. Any of the foregoing examples, such as any of examples 23-40, wherein the ratio of the width to the length includes between 1:1 and 2:1.

Example 42. Any of the foregoing examples, such as any of examples 23-41, wherein the ratio of the width to the length includes between 1.25:1 and 1.75:1.

Example 43. Any of the foregoing examples, such as any of examples 23-42, further comprising the artificial valve formed into the valve seat.

Example 44. Any of the foregoing examples, such as any of examples 23-43, further comprising the artificial valve coupled to the valve seat prior to implantation of the docking station.

Example 45. A method comprising guiding a delivery catheter through a patient to a native aortic valve of the patient; deploying an implant within the native aortic valve, the implant comprising: an inner wire frame forming a valve seat configured to receive or couple to an artificial valve; an outer wire frame joined to the inner wire frame at one or more apices, the outer wire frame comprising a series of cells about a circumference of the outer wire frame; and a sealing skirt that extends from the valve seat to the apices and turns upward along an exterior region of the outer wire frame, wherein a ratio of a width to a length of the docking station includes a ratio greater than 1:1 and less than 3:1; guiding a procedure catheter through the patient to the implant through the outer wire frame, and into an ostia downstream from the native aortic valve; and performing a procedure via the procedure catheter in the ostia.

Example 46. Any of the foregoing examples, such as example 45, wherein the method further comprises guiding a replacement valve through the patient to the implant and deploying the replacement valve within the implant.

Example 47. Any of the foregoing examples, such as any of examples 45-46, wherein the implant includes a replacement valve permanently affixed to the inner wire frame.

Example 48. Any of the foregoing examples, such as any of examples 45-46, wherein the procedure includes a coronary angioplasty.

Example 49. A method comprising: guiding a procedure catheter through a patient to a docking station, the docking station comprising: an inner wire frame forming a valve seat configured to receive or couple to an artificial valve; an outer wire frame joined to the inner wire frame at one or more apices, the outer wire frame comprising a series of cells about a circumference of the outer wire frame; and a sealing skirt that extends from the valve seat to the apices and turns upward along an exterior region of the outer wire frame, wherein a ratio of a width to a length of the docking station includes a ratio greater than 1:1 and less than 3:1; guiding the procedure catheter through the outer wire frame, and into an ostia downstream from the native aortic valve; and performing a procedure via the procedure catheter in the ostia.

Example 50. Any of the foregoing examples, such as example 49, wherein the implant includes a replacement valve in the valve seat of the inner frame.

Example 51. Any of the foregoing examples, such as examples 49-50, wherein the procedure includes a coronary angioplasty.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the following claims. We therefore claim all that comes within the scope and spirit of these claims.

The methods disclosed herein comprise one or more steps or actions to achieve the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions.

The following claims are not intended to be limited to the embodiments shown herein, but are to be accorded the full scope consistent with the language of the claims. Within a claim, reference to an element in the singular is not intended to mean "one and only one" unless specifically so stated, but rather "one or more." Unless specifically stated otherwise, the term "some" refers to one or more. No claim element is to be construed under the provisions of 35 U.S.C. §112(f) unless the element is expressly recited using the phrase "means for" or, in the case of a method claim, the element is recited using the phrase "step for." All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

Unless specific arrangements described herein are mutually exclusive with one another, the various implementations described herein may be combined to enhance system functionality or to produce complementary functions. Likewise, aspects of the implementations may be implemented in standalone arrangements. Thus, the above description has been given by way of example only and modification in detail may be made within the scope of the present invention.

With respect to the use of substantially any plural or singular terms herein, those having skill in the art may translate from the plural to the singular or from the singular to the plural as is appropriate to the context or application. The various singular/plural permutations may be expressly set forth herein for sake of clarity. A reference to an element in the singular is not intended to mean "one and only one" unless specifically stated, but rather "one or more." Moreover, nothing disclosed herein is intended to be dedicated to the public regardless of whether such disclosure is explicitly recited in the above description.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A docking station for use in aortic valve replacement, comprising:
an inner wire frame forming a valve seat configured to receive or couple to an artificial valve;
an outer wire frame joined to the inner wire frame at one or more apices, the outer wire frame comprising a series of cells about a circumference of the outer wire frame; and
a sealing skirt that extends from the valve seat to the apices and turns upward along an exterior region of the outer wire frame,
wherein a ratio of a width to a length of the docking station includes a ratio greater than 1:1 and less than 3:1, and
wherein the sealing skirt turns upward.

2. The docking station of claim 1 wherein the sealing skirt turns upward less than 5 mm.

3. The docking station of claim 2 wherein a distance the sealing skirt turns upward prevents sealing against one or more aortic ostia.

4. The docking station of claim 1, wherein the inner wire frame extends from the apices no further than a downstream end of the outer wire frame.

5. The docking station of claim 1, wherein the inner wire frame is formed of one or more single wires extending from the apices to the valve seat.

6. The docking station of claim 1, wherein a junction between the outer wire frame and the inner wire frame forms a V-shaped cross-section.

7. A method comprising implanting a docking station, the docking station comprising:
an inner wire frame forming a valve seat configured to receive or couple to an artificial valve;
an outer wire frame joined to the inner wire frame at one or more apices, the outer wire frame comprising a series of cells about a circumference of the outer wire frame; and
a sealing skirt that extends from the valve seat to the apices and turns upward along an exterior region of the outer wire frame,
wherein a ratio of a width to a length of the docking station includes a ratio greater than 1:1 and less than 3:1.

8. The method of claim 7, wherein the sealing skirt turns upward less than 5 mm.

9. The method of claim 8, wherein a distance the sealing skirt turns upward prevents sealing against one or more aortic ostia.

10. The method of claim 7, wherein the inner wire frame extends from the apices no further than a downstream end of the outer wire frame.

11. The method of claim 7, wherein the inner wire frame is formed of one or more single wires extending from the apices to the valve seat.

12. The method of claim 7, wherein a junction between the outer wire frame and the inner wire frame forms a V-shaped cross-section.

13. The method of claim 7, wherein a junction between the outer wire frame and the inner wire frame forms a U-shaped cross-section.

14. The method of claim 7, wherein the docking station is crimpable.

15. The method of claim 7, wherein the docking station is self-expanding.

16. The method of claim 7, wherein the docking station is balloon-expandable.

17. A method comprising:
guiding a delivery catheter through a patient to a native aortic valve of the patient;
deploying a docking station within the native aortic valve, the docking station comprising:
an inner wire frame forming a valve seat configured to receive or couple to an artificial valve;
an outer wire frame joined to the inner wire frame at one or more apices, the outer wire frame comprising a series of cells about a circumference of the outer wire frame; and
a sealing skirt that extends from the valve seat to the apices and turns upward along an exterior region of the outer wire frame,
wherein a ratio of a width to a length of the docking station includes a ratio greater than 1:1 and less than 3:1;
guiding a procedure catheter through the patient to the docking station through the outer wire frame, and into an ostia downstream from the native aortic valve; and
performing a procedure via the procedure catheter in the ostia.

18. The method of claim 17, further comprising:
guiding a replacement valve through the patient to the docking station; and
deploying the replacement valve within the docking station.
